(19) United States
(12) Patent Application Publication
(10) Pub. No.: US 2004/0193266 A1
(43) Pub. Date: Sep. 30, 2004

(54) Expansible prosthesis and magnetic apparatus

(76) Inventor: Rudolf Xaver Meyer, Pacific Palisades, CA (US)

Correspondence Address: Rudolf X. Meyer, 16966 Livorno Drive, Pacific Palisades, CA, 90272, US

(21) Appl. No.: 10/401,849

(22) Filed: Mar. 31, 2003

(51) Int. Cl. A61F 2/28

(52) U.S. Cl. 623/16.11

(57) ABSTRACT

The disclosure is for a prosthesis for children and young adults that, once surgically implanted, can be expanded non-invasively later and periodically, as dictated by the growth of the unaffected limbs. It applies particularly to patients at the growing age who suffer from bone cancer or other diseases of the bones of the limbs. Whereas current medical practice requires repeated surgical interventions to periodically implant longer and longer prostheses to keep up with normal growth, this invention requires only a single surgical intervention. Subsequent lengthening of the prosthesis is accomplished by means of a magnetic device that is external to the affected limb and is non-invasive.

### SUMMARY OF THE INVENTION

The present disclosure is for an expansible prosthesis that requires surgery only once. The prosthesis can later be lengthened by the non-invasive application of a magnetic field, generated outside the limb. This magnetic field acts on a magnet that is located in the prosthesis. In turn this magnet, through mechanical means, causes the prosthesis to lengthen.

Depending on the medical application, the two ends of the main body of the prosthesis may consist of stems for attachment to the cut bone ends, or may consist of an artificial joint or part of such a joint, such as a knee, hip, elbow or shoulder joint. In the case where the entire bone must be removed, the main body of the prosthesis would have at each of its ends an artificial joint or a part of such a joint. In the case of limbs that have a pair of bones, such as the fibula and tibia or the ulna and radius, it would also be possible to implant two separate, expansible prostheses attached to the pair. These joints or joint parts are conventional.

This disclosure is also for the apparatus that is external to the limb and generates the magnetic field that interacts with the magnet located in the prosthesis.

### BRIEF DESCRIPTION OF THE DRAWINGS

FIG. 1 is a section and view parallel to the axis of the prosthesis for a femur, with a conventional stem for attachment to the cut bone at one end, and a conventional knee joint at the other end. The figure is drawn with the prosthesis partially expanded. The two arrows starting at the letter A indicate the shortest and longest lengths, respectively, of the prosthesis, specifically the corresponding travel distances of the part of the prosthesis that is located at A. The range indicated by the letter B indicates the length of engagement of the male and female threads.

FIG. 2 is in enlarged form and shows two cross-sections of the spring-loaded ratchet that is needed in some applications. One of the cross-sections is taken perpendicular to the axis of the prosthesis, the other parallel to the axis.

FIG. 3 is in enlarged form and is a cross-section of the screws that are used to attach the main body of the prosthesis to the stem.

FIG. 4 is a cross-section taken parallel to the axis of the prosthesis of a cylindrical, thin membrane that is used to prevent body fluids to enter the mechanism and to restrict lubricants to the interior of the prosthesis. As shown, the membrane is partially folded back on itself Expansion of the prosthesis causes the membrane to unfold.

FIG. 5 is in enlarged form a cross-section of a retaining nut.

FIG. 6 is a cross-section and view, taken parallel to the axis of a prosthesis that is a variant of the prosthesis depicted in FIG. 1. The meaning of the letters A and B is the same as was indicated for FIG. 1.

FIG. 7 is a cross-section and view taken parallel to the axis of the prosthesis, together with the perpendicular view, of the prolonged stem of FIG. 6.

FIG. 8 is a perspective and exploded view of a separator plate.

FIG. 9 is a cross-section and view of a variant of the prostheses shown in FIG. 1 and in FIG. 6. The meaning of the letter A is the same as in FIG. 1 and in FIG. 6.

FIG. 10 is an enlarged cross-section perpendicular to the axis of the prosthesis that is shown in FIG. 9.

FIG. 11 shows three cross-sections of the coupling that in some of the variants of the prosthesis connects the main shaft to the threaded rods that engage the threads inside the stem. One cross-section is along the axis of the prosthesis, and the other two are mutually perpendicular and perpendicular to the axis.

FIG. 12 illustrates schematically the implanting by the surgeon of the type of prosthesis shown in FIG. 9, after having first installed the stem or stems for attachment to the bones.

FIG. 13 is a perspective view of the magnetic apparatus that is external to the limb and that generates the magnetic field that interacts with the magnet in the prosthesis. The illustration is for the case where the prosthesis is located in the femur, close to the knee, but applies with minor modifications also to other prosthesis locations. The arrow C indicates the direction in which the magnetic apparatus can be rotated around the limb.

### DETAILED DESCRIPTION OF THE PREFERRED EMBODIMENT

As shown in the drawings for purposes of illustration, the invention consists of a prosthesis, indicated generally by the reference numbers 10, 20, 30, and 40, together with a magnetic apparatus, indicated by the reference number 50.

In FIG. 1, the illustrative prosthesis 10 has a conventional knee joint 1 with stem 2 that is attached by the surgeon to the bone 3. The cylindrically shaped permanent magnet 4 has a magnetization vector that is in a direction perpendicular to the axis. Under the influence of the rotating magnetic field that is generated externally to the limb, the magnet 4 rotates about its axis. It is attached by adhesive or by other conventional means, such as a key and slot, to the tube 5 that is threaded on its outside over a portion over the length indicated by the letter B. Tube 5 engages a corresponding female thread on the inside of tube 6. This tube is attached at one of its ends to the knee joint 1. The other end is without the female thread, but has a circular groove that accommodates a standard O-ring 7 that forms a seal. Because of the thread, when the magnet 4 and tube 5 rotate, they are displaced longitudinally relative to the knee joint 6. The range of motion, starting from the arbitrary, intermediate position shown in the figure, to either the positions corresponding to the shortest or longest length of the prosthesis is indicated by the letter A.

Also attached to the knee joint 1 is the tube 8 which has a groove or a spline along its length that meshes with a corresponding key or spline in the shaft 9 so that shaft 9 can move longitudinally, but cannot rotate with respect to the knee joint 1. The purpose of this arrangement is to prevent rotation between bone 11 and knee joint 1. In applications where the tissue and muscles surrounding the prosthesis are strong enough to prevent excessive rotation between 11 and 1, tube 8 and shaft 9 can be omitted.

At the end of the prosthesis opposite to the end with the knee joint 1, there is a conventional stem 12 with grooves 13 for better adherence to the bone and is prolonged with cap 14. Screws 15, shown enlarged in FIG. 3, inserted and tightened by the surgeon after the stem 12 has been fixed in the bone, connect the cap 14 to the tube-disc combination 16. In turn, 16 is attached by means of the screw 17 to the shaft 9. Since this shaft engages by a key way or spline with tube 8, the shaft, and with it the stem 12, cannot rotate with respect to the knee joint 1, but only slide longitudinally. The relative rotation between bones 3 and 11 is therefore restricted to the rotation allowed to the knee joint 1. A threaded ring 18, shown enlarged in FIG. 5, is threaded into tube 5. Together with the ring 19 fastened by screw 17 to the shaft 9, they force the longitudinal motion of the non-rotating stem 12 to be the same as the one of the rotating tube 5 and magnet 4. A ring 21, made from Teflon or similar plastic, serves to reduce the friction between the rotating tube 5 and the non-rotating tube-disc combination 16.

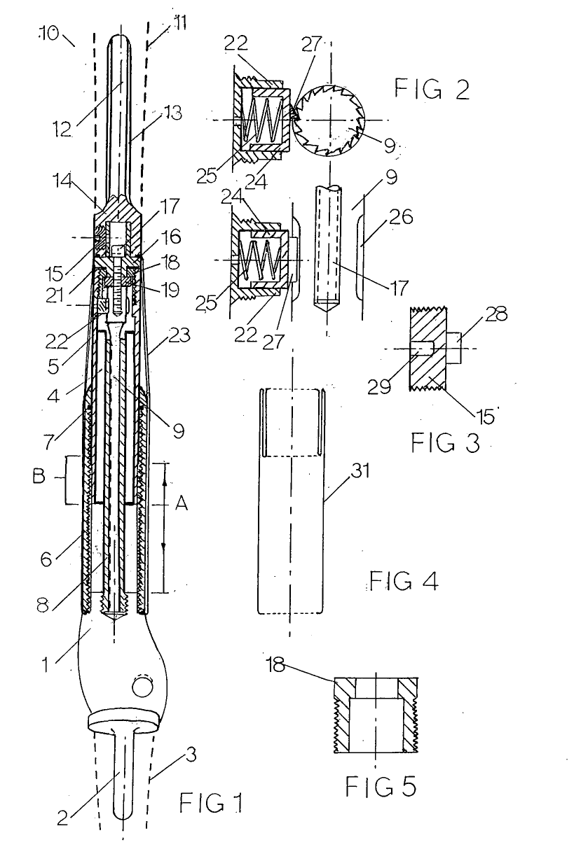

22 is the housing of a ratchet mechanism, shown in FIG. 2 on a large scale. Depending on the character of the loading imposed on the bone of the limb, and depending on the pitch of the thread common to 5 and 6, the ratchet mechanism may be needed to prevent an unintended shortening of the prosthesis.

23 is a cylindrically shaped elastomer surrounding the prosthesis. Depending on the application, it may be needed to prevent body fluids to enter the prosthesis or to prevent lubricants to leave the prosthesis. As the prosthesis lengthens, the elastomer becomes more stretched.

FIG. 2 shows the ratchet mechanism in enlarged form. 22 is its housing which is threaded into tube 5. Piston 24 is pushed by the spring 25 towards the shaft 9 with saw teeth 26 that engage the wedge 27 attached to 24.

FIG. 3 is an enlarged cross-section of the screws 15. The cylindrical part is a close fit with the hole in the tube-disc combination 16. Slot 29 is for insertion by the surgeon of a screwdriver.

FIG. 4 shows a thin membrane 31 which can replace elastomer 23 if needed. In the configuration shown, it is folded back on itself, but unfolds when the prosthesis is expanded.

FIG. 5 is an enlarged cross-section of the threaded ring 18.

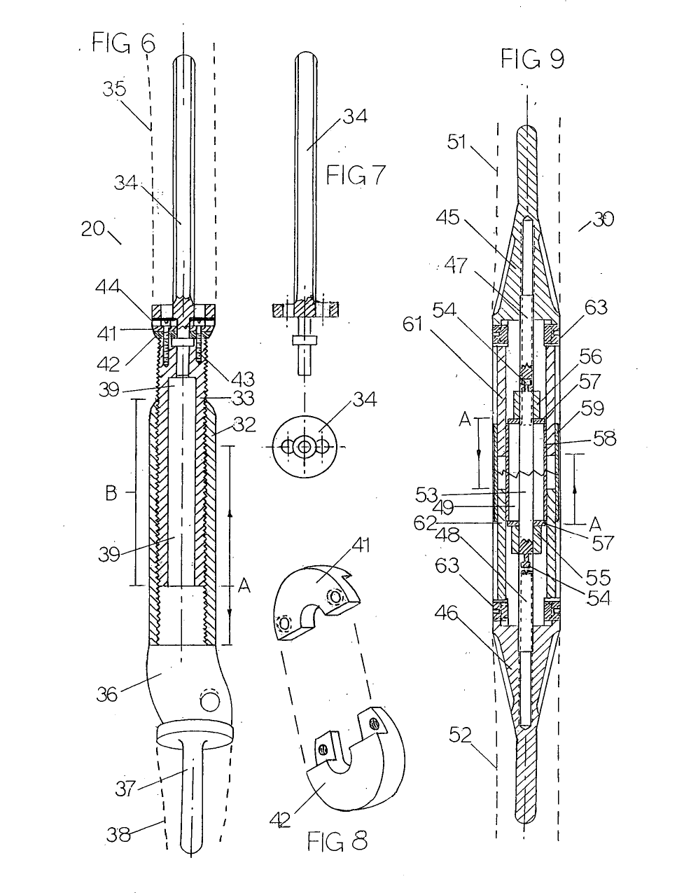

FIG. 6 illustrates the prosthesis 20. It is a different version of prosthesis 10. As shown, the prosthesis is in an intermediate position of expansion. The thread of the outer threaded tube 32 meshes with the thread of the inner threaded tube 33. At the prosthesis expansion shown in the figure, the overlap of the two threads is the distance that is indicated by the letter B. The two arrows starting at the letter A indicate the extreme positions assumed by the end of the threaded tube 33, and therefore indicate the total displacement of the augmented stem 34 and bone 35 relative to the knee joint 36, stem 37 and bone 38.

The permanent magnet 39 is cylindrical and has a magnetization vector perpendicular to its axis. The magnet is fixed by means of an adhesive or keyway and slot or by other such well-known means to the inner threaded tube 33. Rotation of the magnet therefore induces a change in length of the prosthesis.

In this version of the prosthesis there is no shaft that would prevent an arbitrary amount of rotation between bones 35 and 38. Therefore it is only the tissue and muscles that surround the prosthesis and, possibly, an externally applied support that would prevent excessive relative rotation.

A split plate, consisting of two parts 41 and 42, is attached to the threaded tube 33 by the screws 43. This plate needs to be split into two parts so that it can be inserted between the two parts of the augmented stem 34. Its purpose is to prevent stem 34 and bone 35 to be displaced relative to bone 38 by more than is allowed by the rotation of the magnet 39. A disc 44 made from conventional Teflon or other plastic material is inserted for the purpose of reducing the friction between the augmented stem 34 and the split plate.

FIG. 7 shows, for greater clarity, the augmented stem 34 in a cross-section and view along the prosthesis axis and also in a view taken perpendicular to this axis.

FIG. 8 shows the two parts 41 and 42 of the split plate in a perspective and exploded view.

FIG. 9 shows the variant 30 of the prosthesis. Its main feature is the location of female threads inside the stems 45 and 46. These threads engage with the threads on the shaft ends 47 and 48. One of these threads is right handed, the other left handed. Dependent on the chosen sense of rotation of the ring magnet 49 the length of the prosthesis can therefore be made to increase. The magnet 49 is connected by adhesive or keyway and key to the shaft part 53 and is further secured by the sleeve 55, the nut 56 and the disks 57. So that the main body of the prosthesis can be conveniently mounted after the stems 45 and 46 have been first fixed by the surgeon in the bones 51 and 52 the length of the shaft is split into the three parts 47, 48, and 53. When the prosthesis is fully installed, the two shaft couplings 54 connect these parts.

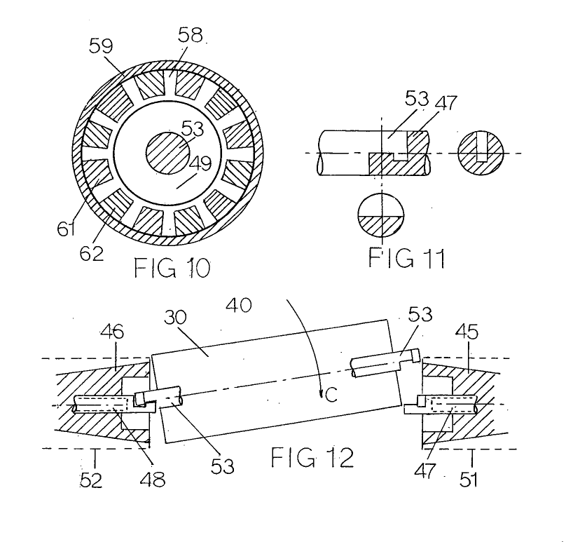

The sleeves 58 and 59 form a cage to guide the extensions 61 and 62, which are more clearly shown in FIG. 10. For reasons of machining, sleeve 59 is shrunk-fit on 58. The two arrows starting at the letters A indicate the travel of the ends of the extensions 61 and 62, starting from the shortest and going to the longest length of the prosthesis. The other ends of 61 and 62 are rigidly attached to the stems 45 and 46 by means of screws 63.

FIG. 10 shows in enlarged form a cross-section of prosthesis 30. The extensions 61 and 62 alternate with each other in the circumferential direction. They can slide longitudinally, but are otherwise constrained by the cage formed by 58 and 59. 49 is the permanent magnet, magnetized in a direction perpendicular to its axis. 53 is the central portion of the main shaft.

FIG. 11 shows cross-sections of the coupling between the shafts 47 and 53. One of the cross-sections is taken along the axis of the prosthesis, the other two are mutually perpendicular and perpendicular to the axis. The same diagram also applies to the coupling between the shafts 48 and 53.

FIG. 12 shows how the prosthesis 30 can be inserted after the stems 45 and 46 have been inserted into the bones 51 and 52 respectively. 53 is the central portion of the main shaft, 47 and 48 the threaded portions of the shaft. The arrow labeled with the letter C indicates the direction of the insertion.

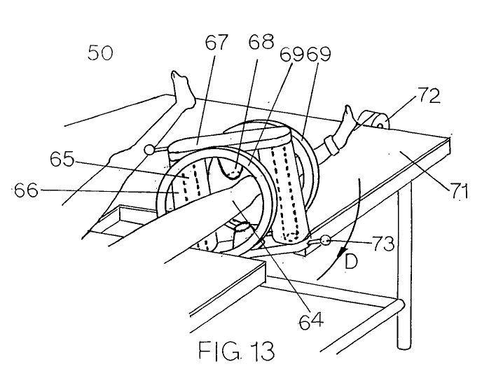

FIG. 13 illustrates in perspective the apparatus, external to the limb, needed to generate the magnetic field that interacts with the magnet in the prosthesis for the purpose of lengthening it periodically as the body grows. In this illustration, the prosthesis replaces part of the femur in the leg 64 and, possibly, also the knee joint. It is understood that virtually the same apparatus is also applicable to other locations of the prosthesis. Two electromagnets with iron cores 65 and windings 66 provide magnetic fluxes, both in the same direction, that are conducted by two plates 67 made from soft iron to the two poles pieces 68, also made from soft iron. The tips of the pole pieces are as close to the limb as practicable. The electromagnetic assembly consisting of 65, 66,67 and 68 can be rotated, either manually by handles 73 or by electric motor in a conventional manner around the limb. The arrow D indicates the direction of rotation for lengthening a particular prosthesis. For this purpose, the assembly is supported by two circular bearings 69. Their non-rotating parts are attached to the table 71, and the rotating parts to the electromagnetic assembly. The current to the electromagnets can be provided from a standard direct current, stationary power supply by a standard slip ring and collectors, or by a cable wound on a drum. Provision is also made on the table 71 for stretching the limb in a controlled manner by means of an assembly 72 consisting of a line, crank, and a standard load measurement device.

While particular forms of the invention have been illustrated and described, it will be apparent that various modifications can be made to the present invention without departing from the spirit and scope thereof The term bone as used in the claims below is meant to apply to any bone shafts or parts thereof of the skeleton and to any of the bones that form the body's joints or parts of such joints.

I claim as my invention:

1. A prosthesis that is surgically implanted and a magnetic apparatus that is external to the patient's body, comprising:
a magnet in the said prosthesis;
a mechanism that transforms the displacement or rotation, relative to other parts of the said prosthesis, of the said magnet into a changed length of the said prosthesis;
means for attaching the said prosthesis to the bones;
means to move relative to the patient's body the magnetic field that is generated by the said magnetic apparatus.

2. The combination defined in claim 1 wherein the said mechanism comprises at least one pair of mutually meshing male and female threads.

3. The combination defined in claim 1 wherein the relative rotation between the attachments to the different bones is substantially prevented by a mechanical link that is a part of the said prosthesis.

4. The combination defined in claim 1 wherein the said magnet in the prosthesis is a permanent magnet.

5. The combination defined in claim 1 wherein the said magnet in the prosthesis is an electromagnet with associated electric power supply and on-and-off switch.

6. The combination defined in claim 1 wherein there is a ratchet mechanism or other equivalent, conventional device that allows an increase of the length of the said prosthesis, but prevents a reduction of its length.

7. The combination defined in claim 2 wherein there is a means that prevents a lengthening of the said prosthesis by more than is allowed by the said pairs of mutually meshing male and female threads when tension is applied to the said prosthesis.

8. The combination defined in claim 1 wherein the said magnetic apparatus is supported by at least one bearing, the bearing's non-rotating parts being attached to a table or other furniture that supports the patient, and the bearing's rotating parts being attached to the said magnetic apparatus.

9. The combination defined in claim 1 wherein the magnetic field of the said magnetic apparatus is generated by at least one electromagnet.

10. The combination defined in claim 1 wherein the magnetic field of the said magnetic apparatus is generated by at least one permanent magnet.

11. The combination defined in claim 9 wherein the pole pieces of the said magnetic apparatus are shaped to maximize the magnetic field strength at the location of the said magnet in the prosthesis.

12. The combination defined in claim 10 wherein the pole pieces of the said magnetic apparatus are shaped to maximize the magnetic field strength at the location of the said magnet in the prosthesis.

13. The combination defined in claim 8 wherein the rotation of the said magnetic apparatus is made manually.

14. The combination defined in claim 8 wherein the rotation of the said magnetic apparatus is produced by an electric motor.

15. The combination defined in claim 1 wherein the said magnetic apparatus generates electrically a rotating magnetic field.

16. The combination defined in claim 1 wherein the said magnetic apparatus comprises a standard Gauss meter used for the observation by the physician of the motion of the said magnet in the prosthesis, obviating thereby the need for repeated x-ray examinations.

17. The combination defined in claim 1 wherein the said magnet in the prosthesis is surrounded by non-magnetic material.

18. The combination defined in claim 1 wherein the said prosthesis is isolated from the surrounding body tissue by an elastomer that is attached to the said prosthesis and where the elastomer stretches as the length of the said prosthesis is increased.

19. The combination defined in claim 1 wherein the said prosthesis is isolated from the surrounding body tissue by a partially folded, flexible sheet that is attached to the said prosthesis and that unfolds as the length of the said prosthesis is increased.

20. The combination defined in claim 1 wherein there is provision made to mechanically stretch or compress the patient's body part that has been operated on.
